(19) United States
(12) Patent Application Publication
(10) Pub. No.: US 2025/0057893 A1
(43) Pub. Date: Feb. 20, 2025

(54) PHARMACEUTICAL COMPOSITION FOR PREVENTING OR TREATING CANCER OR INFLAMMATORY DISEASE

(71) Applicant: ENTEROBIOME INC., Goyang-si, Gyeonggi-do (KR)

(72) Inventors: Jae-Gu SEO, Gimpo-si (KR); Joo-Hyun SHIN, Seoul (KR); Dokyung LEE, Seoul (KR); Yoonmi LEE, Goyang-si (KR)

(73) Assignee: ENTEROBIOME INC., Goyang-si, Gyeonggi-do (KR)

(21) Appl. No.: 18/720,104

(22) PCT Filed: Dec. 16, 2022
(86) PCT No.: PCT/KR2022/020594

§ 371 Date: Jun. 14, 2024

(30) Foreign Application Priority Data
Dec. 16, 2021 (KR) 10-2021-0180844
Jul. 18, 2022 (KR) 10-2022-0088313

(51) Int. Cl. A61K 35/74; A61K 9/00; A61K 9/51; C07K 16/28; C12N 1/20; C12R 1/01

(52) U.S. Cl. CPC A61K 35/74; A61K 9/0056; A61K 9/5176; C07K 16/2803; C07K 16/2818; C07K 16/2827; C12N 1/205; C12R 2001/01

(57) ABSTRACT

A novel Akkermansia muciniphila EB-AMDK39 strain (KCTC 13765BP) and composition containing the novel strain or extracellular vesicles (EVs) derived from the strain are disclosed. The composition can be in a form of pharmaceutical, food, or veterinary composition. The compositions are useful to preventing or treating cancer or inflammatory disease, or ameliorating cancer or inflammatory disease.

### TECHNICAL FIELD

The present invention relates to a pharmaceutical composition for preventing or treating cancer or inflammatory disease, and more particularly, to a pharmaceutical composition for preventing or treating cancer or inflammatory disease that contains extracellular vesicles (EVs) derived from an Akkermansia sp..

### BACKGROUND ART

Cancer is a product of uncontrolled and disordered cell proliferation that occurs due to an excess of abnormal cells, and a malignant tumor leaves its primary site and invades other tissues, where it grows rapidly. Due to this characteristic, malignant tumors threaten life.

In order to treat cancer, various therapeutic approaches have been attempted, including chemotherapy using various anticancer drugs, radiotherapy, and antibody therapy targeting specific molecules involved in cancer. However, in the case of chemotherapy or radiotherapy, it also affects normal cells, causing serious side effects, and cancer cells often acquire resistance to anticancer drugs, resulting in treatment failure or recurrence.

Recent studies have reported that extracellular vesicles play an important role in processes such as intercellular signaling and waste management. Thus, clinical applications of extracellular vesicles have recently been of increasing interest. It is expected that, based on the characteristics of specific extracellular vesicles and target cell membranes, it will be possible to develop therapeutic agents that can specifically treat only diseased cells, including cancer cells, without causing side effects on other normal cells.

Meanwhile, studies on the effects of combination therapy of probiotics, which exhibit in vivo beneficial effects such as immunity strengthening, with immune checkpoint inhibitors, have been emphasized in terms of the development of pharmabiotics, but studies associated therewith in various cancer types still remain insufficient.

### DISCLOSURE

### Technical Problem

The present invention has been conceived with the foregoing background in mind, and an object of the present invention is to provide a pharmaceutical composition for preventing or treating cancer or inflammatory diseases containing pharmabiotics-derived extracellular vesicles.

Another object of the present invention is to provide a health functional food for preventing or ameliorating cancer.

Still another object of the present invention is to provide a veterinary composition or a feed additive for preventing or treating cancer or inflammatory disease.

Yet another object of the present invention is to provide a method of treating cancer in a patient by activating the immune system of the patient using pharmabiotics-derived extracellular vesicles.

### Technical Solution

One aspect of the present invention for achieving the above-described objects is directed to a pharmaceutical composition for preventing or treating cancer or inflammatory disease containing extracellular vesicles (EVs) derived from an Akkermansia sp. and a pharmaceutically acceptable carrier, excipient, or stabilizer.

The Akkermansia sp. may be an Akkermansia muciniphila, preferably an Akkermansia muciniphila EB-AMDK39 strain (KCTC 13765BP).

The pharmaceutical composition for preventing or treating cancer or inflammatory disease according to the present invention may further contain a cancer treatment agent such as a cancer chemotherapeutic agent or a cancer immunotherapeutic agent. The cancer immunotherapeutic agent may be selected from the group consisting of anti-PD1, anti-PDL1, anti-CTLA, anti-Tim3, and anti-LAG3. The extracellular vesicles (EVs) derived from the Akkermansia sp. strain and the cancer chemotherapeutic agent or cancer immunotherapeutic agent may be administered simultaneously in a single dosage form, or may be administered simultaneously or sequentially in separate dosage forms.

Another aspect of the present invention is directed to a health functional food for preventing or ameliorating cancer containing extracellular vesicles (EVs) derived from an Akkermansia sp. and a nutraceutically or dietetically acceptable carrier or excipient.

Yet another aspect of the present invention is directed to a method for treating cancer including administering to a subject a therapeutically effective amount of extracellular vesicles (EVs) derived from an Akkermansla sp. strain.

Still yet another aspect of the present invention is directed to a veterinary composition for preventing or treating cancer or inflammatory disease containing extracellular vesicles (EVs) derived from an Akkermansla sp. and a pharmaceutically acceptable carrier, excipient, or stabilizer.

A further aspect of the present invention is directed to a novel Akkermansia muciniphila EB-AMDK39 strain (KCTC 13765BP).

### Advantageous Effects

The pharmaceutical composition for preventing or treating cancer or inflammatory disease containing extracellular vesicles (EVs) derived from an Akkermansia sp. as an active ingredient according to the present invention may reduce tumor volume, reduce tumor growth, prevent metastasis, or prevent angiogenesis. Thus, it may be developed as an effective anticancer agent.

Even when the pharmaceutical composition for preventing or treating cancer or inflammatory disease containing an Akkermansia sp. according to the present invention is administered alone, it may also exhibit an excellent anticancer effect. However, the pharmaceutical composition is administered in combination with a cancer chemotherapeutic agent or a cancer immunotherapeutic agent, the efficacy thereof may be further activated while the side effects of the cancer chemotherapeutic agent or cancer immunotherapeutic agent are reduced. Thus, co-administration of the pharmaceutical composition and the cancer chemotherapeutic agent or cancer immunotherapeutic agent may exhibit a better anticancer effect compared to when the pharmaceutical composition is administered alone.

The pharmaceutical composition of the present invention has an excellent effect of inhibiting the expression of the pro-inflammatory cytokine genes TNF-α, IL-6 and IL-8, and thus may be useful for preventing or treating inflammatory disease.

### DESCRIPTION OF DRAWINGS

FIG. 1 shows the results of microscopic observation of the Akkermansla muciniphila EB-AMDK39 strain (KCTC 13765BP) of the present invention and the type strain Akkermansia muciniphila ATCC BAA-835 strain.

FIG. 2 shows the results of PCR analysis of the Akkermansia muciniphila EB-AMDK39 strain of the present invention and the Akkermansia muciniphila ATCC BAA-835 strain;

FIG. 3 shows the results of random amplified polymorphic DNA (RAPD) of the genomic DNA of each of the Akkermansia muciniphila EB-AMDK39 of the present invention and the Akkermansia muciniphila ATCC BAA-835 strain;

FIG. 4 shows a phylogenetic tree of the Akkermansia muciniphila EB-AMDK39 strain, prepared based on the 16S rRNA sequence;

FIG. 5 shows the result of examining whether the Akkermansla muciniphila EB-AMDK39 strain of the present invention and the Akkermansla muciniphila ATCC BAA-835 strain have hemolytic activity;

FIGS. 6a and 6b are schematic views showing animal experiments performed in Examples 4 and 5 to evaluate the anticancer activity of the extracellular vesicles (EVs) derived from the Akkermansia muciniphila EB-AMDK39 strain of the present invention;

FIGS. 7 and 8 are graphs showing the anti-oncogenic effect of co-administration of the Akkermansla muciniphila EB-AMDK39 strain of the present invention and anti-PD1 in a syngeneic tumor mouse model, and show time-dependent changes in tumor volume in the syngeneic tumor mouse model;

FIG. 9 shows photographs comparing mouse tumor size between experimental groups for 20 days in a syngeneic tumor animal model in order to evaluate the anticarcinogenic effect of co-administration of the Akkermansia muciniphila EB-AMDK39 strain of the present invention and anti-PD1 in Example 4;

FIG. 10 depicts images showing the results of immunohistochemical staining for CD4, Foxp3, and CD8 in tumors after administration of test substances in Example 5;

FIG. 11 depicts graphs showing the results of immunohistochemical staining for CD4, Foxp3 and CD8 in tumors in Example 5;

FIG. 12 depicts graphs showing changes in tumor size and weight for 25 days when Akkermansia muciniphila EB-AMDK39 strain-derived extracellular vesicles and anti-PD1 were administered in combination to a syngeneic animal model and when anti-PD1 was administered alone to a syngeneic animal model;

FIG. 13 shows photographs comparing mouse tumor size between experimental groups of FIG. 12;

FIG. 14 depicts images showing the results of immunohistochemical staining for CD4, Foxp3, and CD8 in tumors after administration of test substances in Example 7;

FIG. 15 depicts graphs the results of immunohistochemical staining for CD4, Foxp3 and CD8 in tumors in Example 7;

FIG. 16 shows an administration schedule for evaluating the anticancer effect of Akkermansia muciniphila EB-AMDK39 live cells or EVs alone in a syngeneic melanoma mouse model in Example 8;

FIG. 17 is a graph showing B16-F10 tumor growth depending on the concentration of each substance in Example 8;

FIG. 18 is a graph showing tumor weight after administration of each test substance administration in Example 8;

FIG. 19 shows photographs of tumors after administration of each test substance in Example 8;

FIG. 20 depicts images showing the results of immunohistochemical staining for CD4, Foxp3, and CD8 in tumors after administration of test substances in Example 9;

FIG. 21 depicts graphs showing the results of immunohistochemical staining for CD4, Foxp3, and CD8 in tumors in Example 9;

FIGS. 22a and 22b show the results of evaluating the anticancer activity of the Akkermansia muciniphila EB-AMDK39 strain-derived extracellular vesicles of the present invention using wound healing assay in HT29 cells; and

FIG. 23 is a graph comparing the expression of IL-8 mRNA, a pro-inflammatory cytokine, to evaluate the anti-inflammatory effect of Akkermansia muciniphila EB-AMDK39 strain-derived extracellular vesicles (EB-AMDK39 EV) of the present invention.

### MODE FOR INVENTION

Hereinafter, the present invention will be described in more detail with reference to the accompanying drawings.

Unless defined otherwise, all technical and scientific terms used herein have the same meaning as commonly understood by one of ordinary skill in the art to which the present invention pertains.

Throughout the present specification, it is to be understood that when any part is referred to as “including,” “comprising” or “containing” any component, it does not exclude other components, but may further include other components, unless otherwise specified.

As used herein, the term “patient” or “subject” refers to any organism, including a human or non-human, to which the pharmaceutical composition of the present invention is administered for the purpose of preventing or treating cancer. Typical subjects include humans, as well as mice, rats, rabbits, dogs, cats, horses, cows, pigs, deer, and non-human primates.

As used herein, the term “treat,” “treatment” or the like means temporarily or permanently alleviating symptoms, eliminating the cause of symptoms, or preventing or delaying the onset of symptoms of a disease or condition.

As used herein, the term “prevention” refers to any action that suppresses or delays cancer or inflammation or the onset thereof by administration of the pharmaceutical composition according to the present invent ion.

As used herein, the term “alleviation” refers to any action that reduces a parameter associated with an abnormal condition, e.g., the severity of a symptom.

As used herein, the term “pharmaceutically acceptable” means that a composition within the scope of reasonable medical judgment, suitable for use in contact with the tissues of a subject (such as a human), without excessive toxicity, irritation, allergic reaction, or other problems or complications, and with a quite reasonable benefit/risk ratio. Each carrier is “acceptable,” provided that it must be compatible with other formulation ingredients.

As used herein, the term “immune checkpoint inhibitor” refers to a type of drug that blocks certain proteins produced by certain types of cells of the immune system, such as T lymphocytes, and some types of cancer cells, in which these proteins suppress immune response and prevent T lymphocytes from killing cancer cells. “Immune checkpoint inhibitors” well known to date include PD-1/PD-L1 and CTLA-4/B7-1/B7-2.

As used herein, the term “combination therapy” refers to a clinical intervention in which a subject is simultaneously exposed to two or more regimens (e.g., two or more therapeutic agents). In some embodiments, two or more therapeutic regimens may be administered simultaneously. In some embodiments, the two or more therapeutic regimens may be administered sequentially. In some embodiments, the two or more therapeutic regimens are administered in overlapping dosing regimens. In some embodiments, two or more therapeutic agents of a combination therapy are administered to a subject separately, e.g., in separate compositions, via separate administration routes (e.g., one agent orally and another agent intravenously), and/or at different time points. In some embodiments, two or more therapeutic agents may be administered via the same administration route or at the same time.

One aspect of the present invention is directed to a pharmaceutical composition for preventing or treating cancer or inflammatory disease containing extracellular vesicles (EVs) derived from an Akkermansia sp. and a pharmaceutically acceptable carrier, excipient, or stabilizer.

Pharmabiotics are defined as bacterial cells of human origin, or their products, with a proven pharmacological role in health or disease (“Probiotics and pharmabiotics,” Bioeng Bugs. 2010 March-April; 1(2): 79-84.). The pharmaceutical composition of the present invention contains pharmabiotics as an active ingredient, and thus may be safely used without side effects.

The Akkermansia sp. may be an Akkermansia muciniphila. Specifically, the Akkermansia sp. may be an Akkermansia muciniphila EB-AMDK39 strain (KCTC 13765BP).

Extracellular vesicles (EVs) are nano-vesicles of 20 to 300 nm in size that are secreted from cells. These extracellular vesicles contain immunologically important proteins such as the main histocompatibility complex (MHC) and heat shock protein, which induce a strong antitumor immune response. In addition, they contain anti-inflammatory microRNA and microRNA that regulates collagen accumulation.

The Akkermansia sp.-derived extracellular vesicles (EVs) of the present invention may simultaneously exhibit the effects of inhibiting cancer cell proliferation, reducing cancer cell migration and inhibiting angiogenesis, and thus may be used as an excellent anticancer agent. These extracellular vesicles may be administered in combination with a conventional cancer chemotherapeutic agent or cancer immunotherapeutic agent.

The extracellular vesicles (EVs) derived from the Akkermansia sp. strain and the cancer chemotherapeutic agent or cancer immunotherapeutic agent may be administered simultaneously in a single dosage form, or may be administered simultaneously or sequentially in separate dosage forms.

In the present invention, a method for isolating extracellular vesicles is not limited. For example, these extracellular vesicles may be isolated from a culture of the Akkermansia sp. by centrifugation, ultra-high-speed centrifugation, filtration through a filter, gel filtration chromatography, free-flow electrophoresis, capillary electrophoresis, isolation using a polymer, or a combination thereof. Preferably, the extracellular vesicles may be isolated by centrifugation/ultracentrifugation.

In this regard, centrifugation/ultracentrifugation may be performed sequentially at 100 to 300,000×g, preferably 150 to 150,000×g to remove cell debris, non-extracellular vesicle fractions, dead cells, and the like.

Differential centrifugation: The most preferred method for extracellular vesicles is differential centrifugation. This method consists of several steps, is preferably carried out at about 4° C., and includes at least the following steps 1) to 3):
step 1) low-speed centrifugation to remove cells and cell debris;
step 2) high-speed spinning to remove large vesicles >100 nm; and
step 3) high-speed centrifugation to pellet extracellular vesicles.

Density gradient centrifugation: This approach combines ultracentrifugation with a sucrose density gradient. More specifically, density gradient centrifugation is used to separate extracellular vesicles from non-vesicular particles, such as proteins and protein/RNA aggregates. Thus, this method separates vesicles from particles of different densities.

Size exclusion chromatography: Size exclusion chromatography is used to separate macroparticles based on size, not molecular weight. This technique applies a column packed with porous polymer beads containing multiple pores and tunnels. Molecules pass through the beads depending on their diameter. It takes a longer time for molecules with small radii to migrate through pores of the column, while macromolecules elute earlier from the column. Size-exclusion chromatography allows precise separation of large and small molecules.

Filtration: Ultrafiltration membranes may also be used for isolation of exosomes. Depending on the size of microvesicles, this method allows the separation of exosomes from proteins and other macromolecules.

Polymer-based precipitation: Polymer-based precipitation technique usually includes mixing the biological fluid with polymer-containing precipitation solution, incubation at 4° C. and centrifugation at low speed. One of the most common polymers used for polymer-based precipitation is polyethylene glycol (PEG). The precipitation with this polymer has a number of advantages, including mild effects on isolated exosomes and usage of neutral pH.

Isolation by sieving: This technique isolates extracellular vesicles by sieving them from biological liquids via a membrane and performing filtration by pressure or electrophoresis.

The pharmaceutical composition of the present invention has an excellent effect on the prevention or treatment of cancer.

Specifically, the pharmaceutical composition of the present invention may satisfy one or more of the following characteristics:
(a) increasing CD8⁺ T cell levels in tumors;
(b) reducing regulatory T cell levels in tumors; and
(c) increasing macrophage levels in tumors.

Specifically, Akkermansia sp.-derived extracellular vesicles (hereinafter abbreviated as “EB-AMDK39 EV”) are uptaken into cancer cells, inhibit EMT activity, and activate the immune system, thereby inhibiting cancer cell invasion and metastasis. EB-AMDK39 EV plays an important role in regulating T cells to activate innate and adaptive immune systems. In addition, regulatory T cells (Treg), characterized by the expression of Foxp3, are known to suppress anticancer immunity, thereby impeding protective immune surveillance of tumors and hindering effective antitumor immune responses, but EB-AMDK39 EV is presumed to exhibit an anticancer effect by activating T helper cells, thereby activating cytotoxic T cells and suppressing Treg cells. Accordingly, the pharmaceutical composition of the present invention has excellent effects on the prevention, treatment, and suppression of metastasis of cancer.

As used herein, the term “cancer” is meant to include tumors, neoplasias, and malignant tissues or cells. Examples of the cancer include colorectal cancer, lung cancer, small cell lung cancer, gastric cancer, liver cancer, blood cancer, bone cancer, pancreatic cancer, skin cancer, head or neck cancer, skin or intraocular melanoma, uterine cancer, ovarian cancer, rectal cancer, perianal cancer, colon cancer, breast cancer, fallopian tube carcinoma, endometrial carcinoma, cervical cancer, vaginal cancer, vulvar carcinoma, Hodgkin's disease, esophageal cancer, small intestine cancer, endocrine adenocarcinoma, thyroid cancer, parathyroid cancer, adrenal cancer, soft tissue sarcoma, urethral cancer, penis cancer, prostate cancer, chronic or acute leukemia, lymphocytic lymphoma, bladder cancer, kidney cancer, ureteral cancer, renal cell carcinoma, renal pelvic carcinoma, CNS tumor, primary CNS lymphoma, spinal cord tumor, brainstem glioma, pituitary adenoma, or a combination of two or more of these cancers.

In the present invention, the Akkermansia sp.-derived extracellular vesicles (EVs) according to the present invention contained as an active ingredient in the pharmaceutical composition have an average diameter of 20 to 300 nm.

The effective amount of the Akkermansia sp.-derived extracellular vesicles in the pharmaceutical composition according to the present invention may vary depending on the patient's age, sex, and body weight, and may generally be administered daily or every other day or administered 1 to 3 times, at a dose of 0.001 to 150 mg/kg body weight, preferably 0.01 to 100 mg/kg body weight.

The pharmaceutical composition according to the present invention may be used as a single anticancer agent. In addition, the pharmaceutical composition according to the present invention may be used simultaneously, separately or sequentially with radiotherapy, chemotherapy or immunotherapy, if necessary, depending on the circumstances.

The combination therapy of the present invention is intended for use in reducing tumor size or preventing tumor growth in cancer treatment. The combination therapy of the present invention is intended for use in at least one of reducing tumor size, reducing tumor growth, preventing metastasis, or preventing angiogenesis.

Specifically, the pharmaceutical composition of the present invention may be administered as an individual therapeutic agent, or may be in combination with radiotherapy r other therapeutic agents, or may be administered sequentially or simultaneously with conventional radiotherapy or anticancer therapeutic agents. In addition, it may be administered once or multiple times, and it is important to administer the pharmaceutical composition in the minimum amount that may exhibit the maximum effect without side effects, in consideration of all the above factors.

In some embodiments, the cancer therapeutic is an anticancer immunotherapeutic agent. Immunotherapy refers to a treatment that uses a subject's immune system to treat cancer, for example, checkpoint inhibitors, cancer vaccines, cytokines, cell therapy, CAR-T cells, and dendritic cell therapy. Non-limiting examples of immunotherapy are checkpoint inhibitors, including nivolumab (BMS, anti-PD1-1), pembrolizumab (Merck, anti-PD1-1), ipilimumab (BMS, anti-CTLA-4), MEDI4736 (AstraZeneca, anti-PD1-L1), and MPDL3280A (Roche, anti-PD1-L1). Other immunotherapies may be tumor vaccines, such as Gardasil, Cervarix, BCG, sipuleucel-T, Gp100:209-217, AGS-003, DCVax-L, Algenpantucel-L, Tergenpantucel-L, TG4010, ProstAtak, Prostvac-V/RTRICOM, Rindopepimul, E75 peptide acetate, IMA901, POL-103A, Belagenpumatucel-L, GSK1572932A, MDX-1279, GV1001, and Tecemotide. Immunotherapy can be administered via injection (e.g., intravenously, intratumorally, subcutaneously, or into lymph nodes), but may also be administered orally, topically or via an aerosol. Immunotherapy may include adjuvants such as cytokines.

In some embodiments, the immunotherapeutic agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors may be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

Unlike conventional chemotherapy that directly kills cells, immune checkpoint inhibitors are attracting attention as next-generation anticancer agents that have few side effects, such as hair loss, anemia, and suppression of bone marrow function, which reduce the quality of life of cancer patients, compared to anticancer chemotherapy. However, immune checkpoint inhibitors are known to have very low response rates for some cancers (e.g., gastric cancer, colorectal cancer, ovarian cancer, pancreatic cancer, etc.) and cause severe immune-related adverse reactions such as enteritis, hepatitis, pneumonia, hypothyroidism, and pituitary glanditis. It has been reported that the side effects of using immune checkpoint inhibitors mostly appear as minor side effects, but are serious and fatal when they occur rarely in the nervous system or cardiac system. The pharmaceutical composition containing the Akkermansia sp.-derived extracellular vesicles according to the present invention may overcome the response rate limitations of immune checkpoint inhibitors, minimize side effects, and enhance anticancer efficacy.

The pharmaceutical composition of the present invention may inhibit the secretion of cytokine (IL-8) involved in inflammation and allergy, and thus may be useful as a pharmaceutical composition or a cosmetic composition for preventing or treating inflammatory disease.

The inflammatory disease in the present invention may be any one selected from the group consisting of edema, dermatitis, allergy, conjunctivitis, periodontitis, allergic and non-allergic rhinitis, otitis media, sore throat, tonsillitis, pneumonia, gastric ulcer, chronic and acute gastritis, Crohn's disease, colitis, chronic and acute enteritis, hemorrhoids, gout, ankylosing spondylitis, rheumatoiditis, psoriatic arthritis, osteoarthritis, rheumatoid arthritis, shoulder joint inflammation, tendinitis, tenosynovitis, myositis, acute and chronic hepatitis, cystitis, acute and chronic nephritis, Sjogren's syndrome, multiple sclerosis, chronic and acute rhinitis, chronic obstructive pulmonary disease, pulmonary fibrosis, viral infections, bacterial infections, fungal infections, arthritis, Hodgkin's disease, pancreatitis, iritis, scleritis, uveitis, and eczema.

The Akkermansia muciniphila EB-AMDK39 strain (accession number: KCTC 13765BP) of the present invention has the 16S rRNA gene of SEQ ID NO: 1. In addition, a strain closely related to the Akkermansla muciniphila EB-AMDK39 strain, such as a bacterial strain having a 16S rRNA sequence which is 90%, 91%, 92%, 93%, 94%, 95%, 96% or 97% identical to the 16S rRNA sequence of the Akkermansia muciniphila EB-AMDK39 strain, may be used as part of a treatment combination.

The Akkermansla muciniphila EB-AMDK39 strain that is used in the present invention is a mucin-degrading bacterium isolated from healthy Korean feces, which has ellipsoidal cells with an average diameter of 20 to 300 nm, and is a monococcus or diplococcus. It is anaerobic, non-motile, and Gram-negative, and does not form an endospore. The Akkermansla muciniphila EB-AMDK39 strain is capable of producing several mucolytic enzymes, and thus may use mucus as carbon and nitrogen sources. The Akkermansia muciniphila EB-AMDK39 strain may metabolize various carbon sources, including galactose, N-acetylglucosamine, and lactose, and produces, as main metabolites, short-chain fatty acids such as propionic acid and acetic acid.

The pharmaceutical composition for preventing or treating cancer or inflammatory disease according to the present invention may further contain the Akkermansia muciniphila EB-AMDK39 strain. The strain may be selected from among cells of the strain, a lysate of the cells, a culture of the strain, a culture medium obtained by removing cells from the culture of the strain, an extract from the cells of the strain, an extract from the culture of the strain, and an extract from the culture medium obtained by removing cells from the culture of the strain.

The Akkermansia muciniphila EB-AMDK19 strain of the present invention may be recovered by a separation process such as centrifugation, and prepared as a probiotic by drying, for example, freeze-drying, for use. Pasteurization of the Akkermansia muciniphila EB-AMDK19 strain refers to heating at temperature equal to or higher than 50° C. and lower than 100° C. for 10 minutes or more. For example, the strain may be pasteurized at a temperature of 70° C. for 30 minutes.

In one embodiment of the present invention, for use, the pharmaceutical composition containing the Akkermansia muciniphila EB-AMDK39 strain-derived extracellular vesicles according to conventional methods may be formulated into oral preparations such as powders, granules, tablets, capsules, suspensions, emulsions, syrups or aerosols, preparations for external use, suppositories, and sterile injectable solutions, according to the respective conventional methods, without being limited thereto.

In some embodiments, dosage forms of the pharmaceutical composition of the present invention include, but are not limited to, aqueous liquid dispersions, self-emulsifying dispersions, solid solutions, liposomal dispersions, aerosols, solid body forms, powders, immediate release formulations, controlled release formulations, fast melt formulations, tablets, capsules, pills, delayed release formulations, extended release formulations, pulsatile release formulations, multiparticulate formulations, and mixed immediate release and controlled release formulations.

For intravenous, intratumoral or intranasal administration, aqueous suspensions, isotonic saline solutions, or sterile, injectable solutions that contain pharmacologically compatible dispersing agents and/or wetting agents may be used. As an excipient, water, alcohols, polyols, glycerol, vegetable oils, etc., may be used.

The pharmaceutical composition of the present invention may be formulated as a product for enteral or oral administration. In addition, the pharmaceutical composition of the present invention may be productized by enteric coating using any known method so that it can pass through the stomach and then reach the small intestine in which the active ingredient extracellular vesicles (EVs) can be rapidly released into the intestines.

The pharmaceutical composition of the present invention may contain a pharmaceutically acceptable carrier, excipient or a stabilizer, in addition to the active ingredient. In addition, the composition may be formulated with various additives, such as a binder, a disintegrant, a coating agent, and a lubricant, which are commonly used in the pharmaceutical industry.

Pharmaceutically acceptable carriers include, for example, carriers for oral administration or carriers for parenteral administration. Carriers for oral administration include lactose, starch, cellulose derivatives, magnesium stearate, stearic acid and the like. In addition, various drug delivery materials that are used for oral administration may be included. In addition, carriers for parenteral administration include water, suitable oil, saline, aqueous glucose, glycol, and the like. The pharmaceutical composition of the present invention may further contain a stabilizer and a preservative. Suitable stabilizers include antioxidants such as sodium bisulfite, sodium sulfite or ascorbic acid. Suitable preservatives include benzalkonium chloride, methyl- or propyl-paraben, and chlorobutanol. For pharmaceutically acceptable carriers and agents that may be contained in the pharmaceutical composition of the present invention, reference may be made to Remington's Pharmaceutical Sciences, 19^{th} ed., Mack Publishing Company, Easton, PA, 1995.

Excipients that may be used in the present invention include sugars such as sucrose, lactose, mannitol, or glucose; and starches such as corn starch potato starch, rice starch, or partially pregelatinized starch. Binders that may be used in the present invention include polysaccharides such as dextrin, sodium alginate, carrageenan, guar gum, acacia, and agar; naturally-occurring macromolecular substances such as tragacanth, gelatin, and gluten; cellulose derivatives such as hydroxypropylcellulose, methylcellulose, hydroxypropyl methyl cellulose, ethyl cellulose, hydroxypropyl ethyl cellulose, and sodium carboxymethyl cellulose; and polymers such as polyvinylpyrrolidone, polyvinyl alcohol, polyvinyl acetate, polyethylene glycol, polyacrylic acid, polymethacrylic acid, and vinyl acetate resin.

Disintegrants that may be used in the present invention include: cellulose derivatives such as carboxymethylcellulose, calcium carboxymethylcellulose, low-substituted hydroxypropylcellulose, and cellulose derivatives; and starches such as sodium carboxymethyl starch, hydroxypropyl starch, corn starch, potato starch, rice starch, and partially pregelatinized starch.

Examples of lubricants that may be used in the present invention include talc, stearic acid, calcium stearate, magnesium stearate, colloidal silica, hydrous silicon dioxide, and various types of waxes and hydrogenated oils.

Coating agents that may be used in the present invention include, but are not necessarily limited to, water-insoluble copolymers such as a dimethylaminoethyl methacrylate-methacrylic acid copolymer, a polyvinylacetal diethylaminoacetate, an ethylacrylate-methacrylic acid copolymer, an ethylacrylate-methylmethacrylate-chlorotrimethylammonium ethylmethacrylate copolymer, and ethyl cellulose; enteric polymers such as a methacrylic acid-ethyl acrylate copolymer, hydroxypropyl methyl cellulose phthalate, hydroxypropyl methyl cellulose acetate succinate; and water-soluble polymers such as methyl cellulose, hydroxypropyl methyl cellulose, polyvinylpyrrolidone, and polyethylene glycol.

The dosage of the Akkermansia sp.-derived extracellular vesicles as an active ingredient in the pharmaceutical composition for preventing or treating cancer or inflammatory disease according to the present invention may be determined in consideration of various factors, including the type of disease, the patient's age, body weight, sex and medical condition, the severity of the condition, sensitivity to the drug, the duration of administration, the route of administration, the route of administration, excretion rate, and drugs used in combination with the composition, as well as other factors well known in the medical field. Thus, the dose regime can vary widely, but it is important to administer the pharmaceutical composition in the minimum amount that can exhibit the maximum effect without causing side effects, in view of all the above-described factors, and this amount can be easily determined using standard methods by a person skilled in the art.

Another aspect of the present invention is directed to a health functional food containing extracellular vesicles (EVs) derived from an Akkermansia sp. and a nutraceutically or dietetically acceptable carrier or excipient. The extracellular vesicles are preferably extracellular vesicle derived from an Akkermansia muciniphila EB-AMDK39 strain. The health functional food of the present invention may be used to prevent or ameliorate cancer or inflammatory disease.

As used herein, the term “health functional food” is meant to include all forms, including neutraceutical foods, nutritional supplements, health foods, food additive, and feed.

These types of health functional food may be prepared in various forms according to conventional methods known in the art.

General foods include, but are not limited to, beverages (including alcoholic beverages), fruits and their processed foods, fish, meat and their processed foods, bread and noodles, fruit juice, various drinks, cookies, taffy, dairy products, edible plant oils, margarine, vegetable proteins, retort food, frozen food, various sauces, etc., and these foods may be prepared by adding the Akkermansia sp.-derived extracellular vesicles thereto.

In addition, the health functional food of the present invention may further contain various nutrients, vitamins, electrolytes, flavoring agents, colorants, pectic acid or its salt, alginic acid or its salt, organic acids, protective colloidal thickeners, pH adjusting agents, stabilizers, preservatives, glycerin, alcohol, carbonizing agents, or the like.

Still another aspect of the invention is directed to a method of treating cancer in a subject. The method of the present invention includes a step of administering to a subject a therapeutically effective amount of the Akkermansia sp.-derived extracellular vesicles described herein.

Yet another aspect of the present invention is directed to a veterinary composition for preventing or treating cancer or inflammatory disease containing extracellular vesicles (EVs) derived from an Akkermansla sp. and a pharmaceutically acceptable carrier, excipient, or stabilizer. Here, the animal is not particularly limited, and may refer to pets such as dogs, cats, guinea pigs, hamsters, rats, mice, ferrets, rabbits, and the like. The veterinary composition may be a veterinary drug or feed additive.

The present invention provides a novel Akkermansia sp. EB-AMDK39 strain. The strain was deposited with the Korean Collection for Type Cultures (KCTC), the Korea Research Institute of Bioscience and Biotechnology, on Dec. 5, 2018 under accession number KCTC 13765BP.

Hereinafter, the present invention will be described in more detail with reference to examples. However, these examples are merely to illustrate the present invention, and the scope of the present invention is not limited to these examples.

### EXAMPLES

### Example 1: Isolation and Identification of Akkermansla muciniphila EB-AMDK39 Strain

### 1.1. Isolation and Identification of Strain

In order to isolate an Akkermansia sp. from the feces of a healthy Korean (female, 7 years old, BMI: 19.9), according to the method of Derrien, selective culture was performed using mucin medium (0.4 g/L monopotassium phosphate, 0.53 g/L sodium dichlorophosphate, 0.3 g/L sodium chloride, 0.3 g/L aluminum chloride, 0.1 g/L magnesium chloride, 0.11 g/L calcium chloride, 4.0 g/L sodium bicarbonate, 1 mL acidic trace element solution, 1 mL alkaline trace element solution, 1 mL vitamin solution, 2.5 g/L porcine gastric fluid (Type III)), and 0.25 g/L sodium sulfide nonahydrate), and then a strain was isolated (Derrien et al., 2004, Akkermansla muciniphila gen. nov., sp. nov., a human intestinal mucin-degrading bacterium, International Journal of Systematic and Evolutionary Microbiology, 54: 1469-1476).

### 1.2. Microscopic Observation

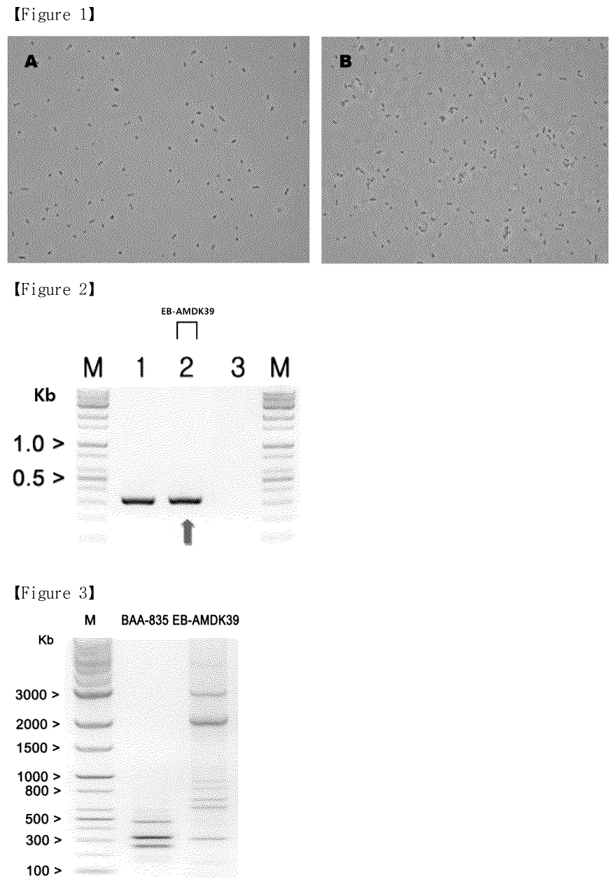

In order to confirm that the isolated strain would be an Akkermansla sp. strain, the isolated strain was observed under a microscope. In FIG. 1, A is a photograph at 1,000× magnification of the type strain Akkermansla muciniphila ATCC BAA-835 strain, and B is a micrograph at 1,000× magnification of an Akkermansia sp. EB-AMDK39 strain. As shown in FIG. 1, as a result of observing the type strain Akkermansia muciniphila ATCC BAA-835 strain (A) and the Akkermansia muciniphila EB-AMDK39 strain (B) at 1,000× magnification, it was confirmed that cells of the two strains had a straight or curved rod shape, indicating that the strains had a similar shape.

### 1.3. PCR Analysis

In order to confirm that the isolated strain would be an Akkermansla sp. strain, the isolated strain was subjected to PCR analysis performed using AM-specific primers (SEQ ID NOS: 2 and 3), and the results of the analysis are shown in FIG. 2. In FIG. 2, M represents a DNA size marker, lane 1 represents a positive control (ATCC BAA-835), lane 2 represents the Akkermansia muciniphila EB-AMDK39 strain, and lane 3 represents a negative control (distilled water).

As shown in FIG. 2, it could be confirmed that the Akkermansla muciniphila EB-AMDK39 strain of the present invention showed a band similar to that of the type strain Akkermansia muciniphila ATCC BAA-835.

TABLE 1

| | | | Amplicon | |
|---|---|---|---|---|
| Designation | Direction | Sequence (5′→3′) | size | SEQ ID NO. |
| AM1 | Forward | CAG CAC GTG AAG GTG | 329 bp | SEQ ID NO: 2 |
| | | GGG AC | | |
| AM2 | Reverse | CCT TGC GGT TGG CTT | | SEQ ID NO: 3 |
| | | CAG AT | | |

### 1.4. Random Amplified Polymorphic DNA (RAPD) Analysis

In order to verify whether the Akkermansia muciniphila EB-AMDK39 strain isolated as described above is the same as the type strain Akkermansia muciniphila ATCC BAA-835 of the same kind as previously reported, random amplified polymorphic DNA (RAPD) printing, which is a kind of molecular typing, was performed. To this end, the genomic DNA extracted from the strain was amplified using the universal primers shown in Table 2 below and was electrophoresed on 1% agarose gel for 1 hour and 30 minutes, and DNA fragmentation patterns were compared on a UV perforator. The results are shown in FIG. 3.

TABLE 2

| Designation | Direction | Sequence (5′→3′) | SEQ ID NO. |
|---|---|---|---|
| ERIC-1 | Forward | ATG TAA GCT CCT GGG GAT TCA C | SEQ ID NO: 4 |
| ERIC-2 | Reverse | AAG TAA GTG ACT GGG GTG AGC G | SEQ ID NO: 5 |

As can be seen in FIG. 3, the Akkermansia muciniphila EB-AMDK39 strain of the present invention showed an RAPD band pattern different from that of the type strain Akkermansia muciniphila ATCC BAA-835. It is known that the RAPD band patterns of Akkermansia muciniphila species are different from each other when the species are different. Thus, it was confirmed that the Akkermansia muciniphila EB-AMDK39 strain of the present invention belongs to the same species as the type strain Akkermansia muciniphila ATCC BAA-835, but was a different strain.

### 1.5. Phylogenetic Tree Analysis Using Full-Length 16S rRNA Gene Sequence

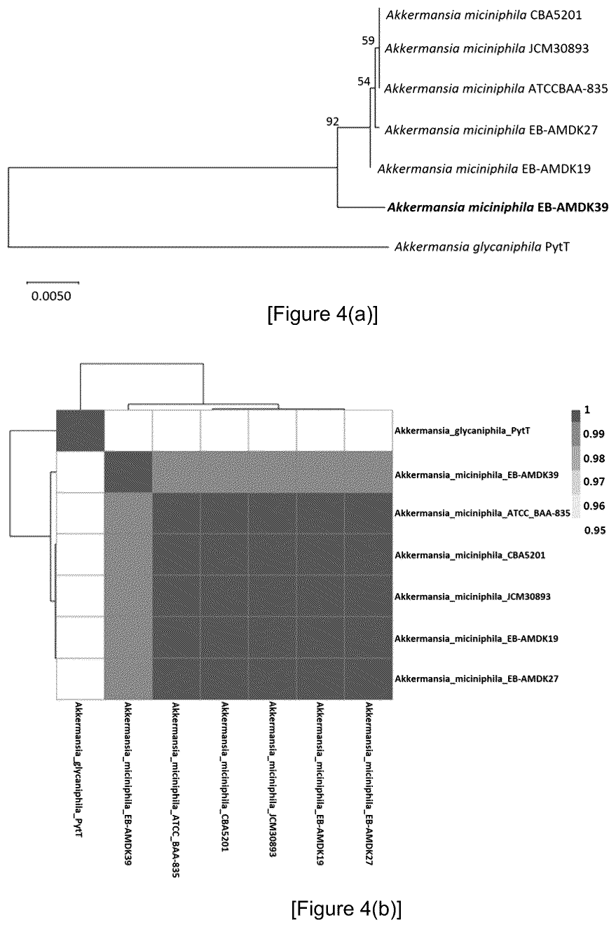

In order to analyze the full-length 16S rRNA gene sequence of the Akkermansia muciniphila EB-AMDK39 strain isolated as described above, the 16S rRNA gene was amplified using the 27F and 1541R primers shown in Table 3 below, and then sequenced using a 3730X1 DNA analyzer. A phylogenetic tree was prepared using the 16S rRNA gene sequences of the Akkermansia muciniphila EB-AMDK19 strain obtained as described above and other strains of the Akkermansia species already published. Phylogenetic analysis was performed using MEGA-X, and a phylogenetic tree was prepared through a neighbor-joining method using 1,000 bootstraps, and is shown in FIG. 4(A). Based on average nucleotide identity (ANI) values, evolutionary distances were evaluated using the pyani v0.2.7 program with “-m ANIb” setting. The complete or draft genome sequences of an Akkermansia muciniphila ATCC BAA-835 strain (accession number: GCF_000020225.1), an Akkermansia muciniphila CBA5201 strain (accession number: GCF_004104435.1), an Akkermansia muciniphila JCM30893 strain (accession number: GCF_009731575.1), an Akkermansla muciniphila EB-AMDK19 strain (accession number: GCF_004015105.1), an Akkermansia muciniphila EB-AMDK27 strain (accession number: GCF_010223015.1) and an Akkermansla glycaniphila PytT strain (accession number: GCF_900097105.1) were downloaded from the NCBI genome database (https://www.ncbi.nlm.nih.gov/genome/) and used. A phylogenetic tree was constructed using the 16S rRNA gene sequences of other strains of the same species and is shown in FIG. 4(B).

TABLE 3

| | | | Amplicon | |
|---|---|---|---|---|
| Designation | Direction | Sequence (5′→3′) | size | SEQ ID NO. |
| 27F | Forward | AGA GTT TGA TCM TGG | 1,505 bp | SEQ ID NO: 6 |
| | | CTC AG | | |
| 1541R | Reverse | AAG GAG GTG ATC CAG | 1,505 bp | SEQ ID NO: 7 |
| | | CCG CA | | |

As shown in FIGS. 4(a) and 4(b), as a result of analyzing the evolutionary relationship between the 16s rRNA gene sequences through the phylogenetic tree, it was confirmed that the Akkermansia muciniphila EB-AMDK39 strain was a strain that genetically belongs to Akkermansla muciniphila species.

The Akkermansia muciniphila EB-AMDK39 strain isolated from human feces was identified through the biochemical method (API) and molecular biological methods (16s rRNA sequencing, 16S rRNA BLAST analysis, and RAPD) using the type strain Akkermansia muciniphila (ATCC BAA-835) as a control. In addition, through the antibiotic resistance test described below, the isolated Akkermansia muciniphila EB-AMDK39 strain was found to be a safe strain that can function as probiotics. Based on these results, the isolated Akkermansia muciniphila strain was named Akkermansia muciniphila EB-AMDK39 strain and deposited with the Korean Collection for Type Cultures (KCTC) under accession number KCTC 13765BP.

### Example 2: Analysis of Mycological Characteristics and Safety of Akkermansia muciniphila EB-AMDK39 Strain

### 2.1. Analysis of Utilization of Carbohydrates by Isolated Akkermansia Sp. Strain

In order to examine the utilization of carbohydrates by the isolated Akkermansia muciniphila EB-AMDK39 strain of the present invention, the strain was cultured using an API50CH kit (Biomerieux, France), and then whether the strain would grow using each carbohydrate was compared with the type strain (ATCC BAA-835). The results of the comparison are shown in Table 4 below.

TABLE 4

| | | ATCC | EB- |
|---|---|---|---|
| No. | Carbohydrates | BAA-835 | AMDK39 |
| 0 | Negative control | − | − |
| 1 | Glycerol | − | − |
| 2 | Erythritol | − | − |
| 3 | D-Arabinose | w | w |
| 4 | L-Arabinose | w | w |
| 5 | Ribose | + | w |
| 6 | D-Xylose | w | w |
| 7 | L-Xylose | w | w |
| 8 | Adonitol | − | − |
| 9 | β-Methyl-xyloside | − | − |
| 10 | D-Galactose | w | − |
| 11 | D-Glucose + Mucin | + | + |
| 12 | D-Fructose | w | − |
| 13 | D-Mannose | + | − |
| 14 | L-Sorbose | − | − |
| 15 | L-Rhamnose | − | − |
| 16 | Dulcitol | − | − |
| 17 | Inositol | − | − |
| 18 | D-Mannitol | − | − |
| 19 | D-Sorbitol | − | − |
| 20 | Methyl-α D- | − | − |
| | mannopyranoside | | |
| 21 | Methyl-α D- | − | − |
| | glucopyranoside | | |
| 22 | N- | + | + |
| | Acetylglucosamine | | |
| 23 | Amygdaline | − | − |
| 24 | Arbutine | − | − |
| 25 | Esculine | − | − |
| 26 | Salicine | − | − |
| 27 | D-cellobiose | − | − |
| 28 | D-Maltose | − | − |
| 29 | D-Lactose | + | − |
| | (bovine origin) | | |
| 30 | D-Melibiose | − | − |
| 31 | D-Saccharose | − | − |
| | (sucrose) | | |
| 32 | D-Trehalose | − | − |
| 33 | Inuline | − | − |
| 34 | D-Melezitose | − | − |
| 35 | D-Raffinose | − | − |
| 36 | Amidon (starch) | − | − |
| 37 | Glycogene | − | − |
| 38 | Xylitol | − | − |
| 39 | Gentiobiose | − | − |
| 40 | D-Turanose | − | − |
| 41 | D-Lyxose | w | w |
| 42 | D-Tagatose | − | − |
| 43 | D-Fucose | − | − |
| 44 | L-Fucose | + | w |
| 45 | D-Arabitol | − | − |
| 46 | L-Arabitol | − | − |
| 47 | Potassium | − | − |
| | Gluconate | | |
| 48 | Potassium 2- | − | − |
| | Ketogluconate | | |
| 49 | Potassium 5- | w | w |
| | Ketogluconate | | |

+: growth,
w: weak growth,
−: no growth

As can be seen in Table 4 above, it was confirmed that the Akkermansla muciniphila EB-AMDK39 strain of the present invention differed from the type strain (ATCC BAA-835) with respect to the utilization of ribose, D-galactose, D-fructose, D-mannose, D-lactose and L-fucose.

### 2.2. Analysis of Antimicrobial Susceptibility of Isolated Strain

In order to examine the antimicrobial susceptibility of the Akkermansia muciniphila EB-AMDK19 strain isolated as described above, the minimum inhibitory concentrations (MICs) of antibiotics for anaerobic bacteria (piperacillin-tazobactam (PTZ), ceftizoxime (CTZ), chloramphenicol (CHL), clindamycin (CLI), meropenem (MEM), moxifloxacin (MXF), metronidazole (MTZ), and ciprofloxacin (CIP)) against the isolated strain were determined by broth microdilution according to the guideline of Clinical & Laboratory Standard Institute (CLSI, 2017), and the results are shown in Table 5 below.

TABLE 5

| | MICª Breakpoints | | | QC | Test strains | |
|---|---|---|---|---|---|---|
| Anti- | (μg/m [symbol] ) | | | ATCC | ATCC | EB- |
| biotics | S | I | R | 297415^{b} | BAA-835 | AMDK39 |
| PTZ | ≤32/4 | 64/4 | ≥128/4 | 8/4 | ≤0.5/4 (S) | ≤0.5/4 (S) |
| CTZ | ≤32 | 64 | ≥128 | 16 | 2 (S) | 256 (R) |
| CHL | ≤8 | 16 | ≥32 | 8 | 4 (S) | 8 (S) |
| CLI | ≤2 | 4 | ≥8 | 4 | ≤0.125 (S) | >64 (R) |
| MEM | ≤4 | 8 | ≥16 | 0.5 | 1 (S) | 4 (S) |
| MXF | <2 | 4 | ≥8 | 8 | >32 (R) | >32 (R) |
| MTZ | ≤8 | 16 | ≥32 | 2 | ≤0.25 (S) | 1 (S) |
| CIP | ≤1 | 2 | ≥4 | >32 | >32 (R) | >32 (R) |

PTZ: Piperacillin-tazobactam,
CTZ: ceftizoxime (3^{rd} gen),
CHL: chloramphenicol,
CLI: clindamycin,
MEM: meropenem,
MXF: moxifloxacin (4^{th} gen),
MTZ: metronidazole,
CIP: ciprofloxacin (2^{nd} gen),
^{a}MIC: minimal inhibitory concentration,
^{b}Bacteroides thetiotaomicron ATCC 29741

As can be seen in Table 5 above, the Akkermansia muciniphila EB-AMDK39 strain of the present invention exhibited resistance to moxifloxacin and ciprofloxacin, which were fluoroquinolone-based antibiotics, and showed susceptibility to the antibiotics except for moxifloxacin and ciprofloxacin. Compared to the type strain, there was some difference in the antimicrobial resistance pattern. It is considered that the resistance of the strain to the fluoroquinolone-based antibiotics is intrinsic resistance that exists in the same Akkermansia muciniphila.

In addition, whether the Akkermansia muciniphila EB-AMDK39 strain has acquired/intrinsic resistance was examined using bioinformatics-based PlasmidFinder (https://cge.cbs.dtu.dk/services/PlasmidFinder/), PHASTER (https://phaster.ca/) and Mobile Element Finder (cge.cbs.dtu.dk/services/MobileElementFinder) programs for the full-length genome of the strain, and as a result, no plasmid existed in the Akkermansla sp. EB-AMDK39 strain, and acquired/intrinsic antibiotic resistance-related genes were not detected at all. Therefore, it could be confirmed that the Akkermansia sp. EB-AMDK39 strain according to the present invention is a safe strain.

### 2.3. Analysis of Hemolytic Activity and Virulence Factors of Isolated Strain

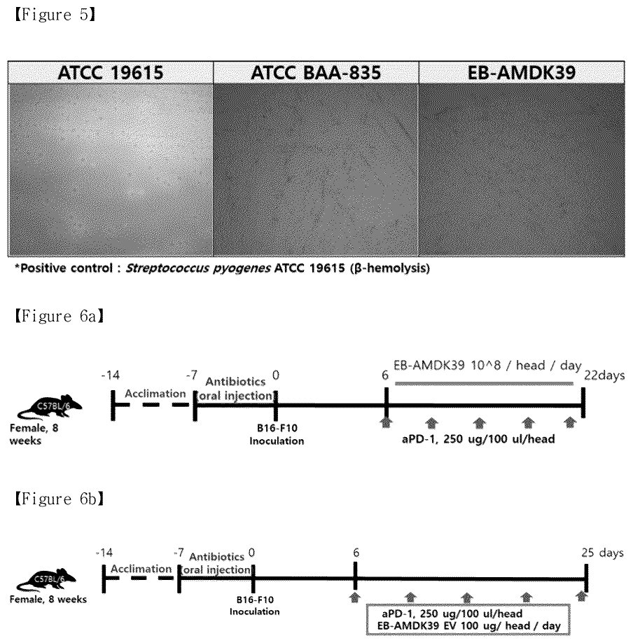

In order to verify the safety of the Akkermansia muciniphila EB-AMDK39 strain isolated as described above, whether the strain would have hemolytic activity was evaluated. To this end, the strain was cultured using a blood agar medium prepared by adding 5% w/v defibrinated sheep blood to tryptic soy agar (17.0 g/L pancreatic digest of casein, 3.0 g/L pancreatic digest of soybean, 2.5 g/L dextrose, 5.0 g/L sodium chloride, 2.5 g/L potassium phosphate, and 15 g/L agar). The results of the culture are shown in FIG. 5. As can be seen in FIG. 5, β-hemolysis (a fully transparent part around a colony) associated with pathogenicity was not observed in the Akkermansla muciniphila EB-AMDK39 strain of the present invention.

Based on VFDB (reference database for bacterial virulence factors, http://www.mgc.ac.cn/VFs), genes encoding virulence factors were analyzed on the full-length genome of the Akkermansia muciniphila EB-AMDK39 strain of the present invention. Analysis was performed under the conditions of protein identity of at least 80%, coverage of at least 80%, and alignment length of at least 50 bp. As a result of the analysis, no virulence factors were detected in the Akkermansia sp. EB-AMDK39 strain. This suggests that the Akkermansia muciniphila EB-AMDK39 strain is harmless to the human body.

### 2.4. Evaluation of the Ability of Akkermansia muciniphila Strain to Produce Short-Chain Fatty Acids (SCFAs)

Short-chain fatty acids (SCFAs), such as butyrate, acetate, and propionate, are metabolites produced by gut bacteria and play an important role in host energy metabolism. They are signaling mediators acting on G protein-coupled receptors (GPR41 and GPR43) and are involved in energy balance.

Short-chain fatty acids (SCFAs) decrease intestinal motility and increase intestinal transit rate, through GPR41 in enteroendocrine cells. Thereby, SCFAs induce PYY (peptide YY) secretion to reduce energy intake and prevent obesity. In addition, GPR43 by short-chain fatty acids induces GPL-1 (glucagon-like peptide 1) to increase insulin sensitivity, thereby increasing satiety, and the activity of GPR43 inhibits insulin signaling in adipose tissue to prevent fat accumulation. Short-chain fatty acids (SCFAs) can enhance glucose metabolism and activate intestinal gluconeogenesis (IGN), which can reduce food intake through the gut-brain neural circuit. Additionally, vitamin B12 affects the ability of the strain to produce short-chain fatty acids. Additionally, vitamin B12 affects the ability of the strain to produce short-chain fatty acids. Specifically, it can act as a coenzyme that catalyzes the conversion of succinic acid to propionic acid. The Akkermansia muciniphila strain of the present invention has methylmalonyl-coA mutase that catalyzes the conversion of succinic acid to propionic acid using vitamin B12 as a cofactor.

In the above Example, the gene cluster, which is present specifically in the Akkermansia muciniphila EB-AMDK39 strain and plays a direct role in vitamin B12 synthesis, was confirmed. Therefore, in order to identify changes in functional metabolites in the Akkermansla muciniphila strains depending on the presence or absence of vitamin B12 during culture, the content of short-chain fatty acids (SCFAs) contained in a culture of the strain was analyzed by gas chromatography (GC) after culturing in a test tube. To this end, the culture was centrifuged at 12,000×g for 5 minutes, and the supernatant was collected, filtered through a 0.2 μm syringe filter, and then used for analysis. Analysis was performed using gas chromatography (Agilent 7890N) equipped with a FFAP column (30 m×0.320 mm, 0.25 μm phase) under the conditions were shown in Table 6 below, and the results of the analysis are shown in Table 7 below.

TABLE 6

| Flow | H₂: 40 ml/min, Air: 350 ml/min |
|---|---|
| Injector temp. | 240° C. |
| Detector temp. | 250° C. |
| Oven temp. | 40° C. (hold 2 min)→ 65° C./10 min |
| | (hold 2 min)→ 240° C./10 min (hold 5 min) |
| Injection vol. | 2 [symbol] |
| Split ratio | 20:1 |

TABLE 7

| | Addition of | Short-chain fatty acid production (μg/mL) | | |
|---|---|---|---|---|
| Strain | vitamin B12 | Acetate | Propionate | Succinate |
| ATCC BAA- | None | 280.64 ± 32.21 | 32.44 ± 14.25 | 785.09 ± 32.41 |
| 835 | 0.1 mg/L | 653.4 ± 78.84 | 1415.97 ± 145.01 | 7.23 ± 6.98 |
| EB-AMDK39 | None | 618.14 ± 79.15 | 1433.01 ± 211.19 | 20.84 ± 28.41 |
| | 0.1 mg/L | 606.09 ± 75.30 | 1429.03 ± 165.42 | 10.20 ± 28.50 |

As can be seen from Table 7 above, the Akkermansia muciniphila EB-AMDK39 strain of the present invention exhibited a short-chain fatty acid production ability different from that of Akkermansia muciniphila ATCC BAA-835 (type strain). Specifically, it was confirmed that, when vitamin B12 was not added to the culture medium, the main short-chain fatty acids produced by Akkermansia muciniphila ATCC BAA-835 were acetic acid and succinate, whereas the Akkermansia muciniphila EB-AMDK39 strain of the present invent ion produced acetate and succinate as main short-chain fatty acids regardless of the presence or absence of vitamin B12, and in particular, the production of propionic acid produced by the Akkermansia muciniphila EB-AMDK39 strain was about 40 times higher than that produced by the type strain. From these results, it can be seen that the Akkermansia muciniphila EB-AMDK39 strain of the present invention has a clear difference in short-chain fatty acid production ability from the type strain Akkermansia muciniphila ATCC BAA-835 depending on the presence or absence of vitamin B12.

### Example 3: Isolation of Extracellular Vesicles from Akkermansia Muciniphila Strain

To obtain extracellular vesicles (EVs) from the Akkermansia muciniphila EB-AMDK39 strain, a culture of the Akkermansia muciniphila EB-AMDK39 strain was centrifuged at 10,000×g at 4° C. for 20 minutes, and the supernatant was collected and filtered through a 0.45 μm filter and a 0.22 μm filter. The filtered supernatant was subjected to high-speed centrifugation at 150,000×g at 4° C. for 2 hours to obtain a pellet, which was then dissolved in sterile phosphate buffered saline (PBS) and used for protein quantification and then efficacy testing.

### Example 4: Anticancer Effect of Co-Administration of Cancer Immunotherapeutic Agent aPD-1 and Akkermansia muciniphila EB-AMDK39 Live Cells in Syngeneic Melanoma Mouse Animal Model

### 4.1. Strain Sample

Akkermansia muciniphila EB-AMDK39 (KCTC 13765BP) live cells used in this experiment were prepared at a concentration of 1×10⁸ CFU/150 μl PBS (25% glycerol, 0.05% cysteine/PBS).

### 4.2. Animal Experiments

Animal experiments were conducted in compliance with the Animal Use and Care Protocol of the Institutional Animal Care and Use Committee (IACUC). For cancer induction, 8-week-old female C57BL/6 mice were purchased and acclimated for 1 week. Then, the mice were bred for 12 weeks. During breeding, the animals were kept at a constant temperature of 22° C. and a relative humidity of 40 to 60% with 12-hr light/12-hr dark cycles.

To prepare a syngeneic tumor animal model, mouse-derived melanoma cells (B16-F10) were used.

The syngeneic model is a technique in which a mouse cell line grown in vitro is transplanted into and grown in an actual mouse and grown, and the identical host and cell line strain mean that tumor rejection does not occur.

After 1 week of acclimation, the mice were pretreated with the antibiotics shown in Table 8 below for 1 week.

TABLE 8

| Antibiotic | Concentration |
|---|---|
| Ampicillin (Sigma-A0166) | 1 g/L |
| Vancomycin (Sigma SBR00001) | 0.5 g/L |
| Metronidazole (Sigma M1547) | 1 g/L |
| Neomycin (Sigma N6386) | 1 g/L |
| Amphotericin B (Sigma PHR1662) | 0.1 g/L |

Subsequently, 2×10⁴ B16-F10 cells were subcutaneously (SC) injected into the thigh of each mouse together with 100 μl of Matrigel.

### 4.3. Sample Administration and Experimental Group Setup

Each drug shown in Table 9 below was orally administered to mice every day for 5 weeks. As a positive control, anti-PD1 antibody was orally administered into each mouse at a concentration of 250 μg/100 μl.

Cancer cells began to appear on day 6, and from this time point, the Akkermansia muciniphila EB-AMDK39 strain was orally administered daily at 10⁸ CFU. From day 6, 250 μg of aPD-1 antibody was intraperitoneally injected every 4 days. In this case, as the aPD-1 antibody, InVivoMab anti-mouse PD-1 (RMP1-14) (catalog #BE0146, BioX Cell) was used, which was diluted with InVivoPure pH 7.0 dilution buffer (catalog #IP0070) at a concentration of 250 μg/100 μl.

During tumor growth, the mice were weighed twice a week and monitored daily, and from day 6 when the cancer cells appeared, the tumor size was monitored every other day using a computerized caliper (see FIG. 6a).

Tumor volume was calculated according to the following equation by measuring the two diameters (major and minor diameters) of each tumor.

$$Tumor volume ( {mm}^{3} ) = [ major diameter \times minor {diameter}^{2} ] / 2 \qquad [ Equation 1 ]$$

TABLE 9

| Group | Experimental group | Drug administered |
|---|---|---|
| I | Control group | PBS |
| II | Positive control group | 250 μg/100 [symbol] /head |
| | (anti-PD1-administered group) | |
| III | Anti-PD1 + A.m. EB-AMDK39 | EB-AMDK39 live cells |
| | strain-administered group | (1 × 10⁸ CFU) + |
| | | anti-PD1 (250 μg/100 [symbol] /head) |

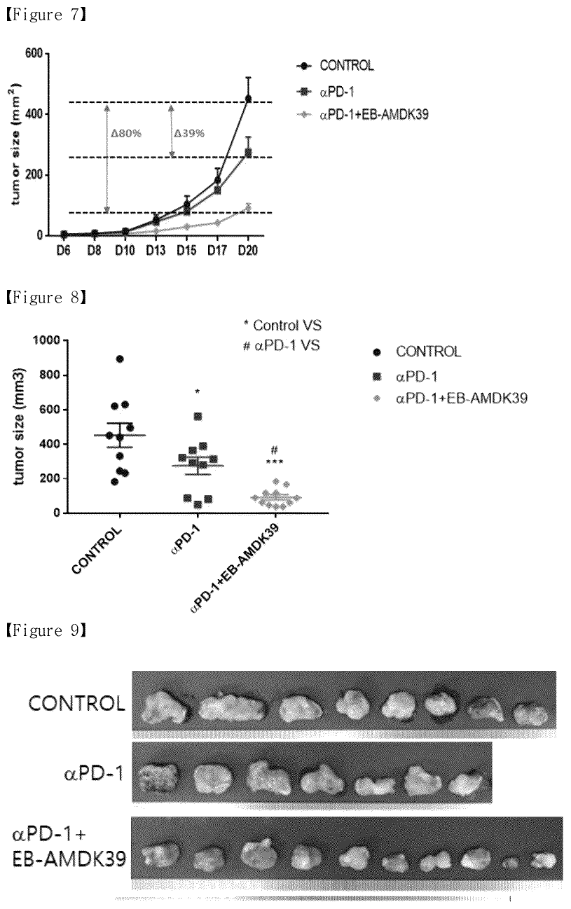

Referring to FIGS. 7 to 9, it was confirmed that the tumor size became significantly smaller in the group to which the Akkermansia muciniphila EB-AMDK39 live cells were orally administered than in the control group into which the B16-F10 cells were syngeneically transplanted. Compared to the tumor size in the control group (452.4±69.7 mm³), the tumor size in the aPD-1-administered group was 274.7±50.7 mm³ (80% decrease) (p<0.05), and the tumor size in the group to which aPD-1 and the Akkermansia muciniphila EB-AMDK39 strain were co-administered was 91.5±15.19 mm³ (39% decrease) (P<0.001), which significantly decreased. In particular, compared to the aPD-1-administered group, the group to which aPD-1 and EB-AMDK39 were co-administered showed a significant effect of reducing the tumor size by about 67% (p<0.05).

### Example 5: Changes in Immune Cells in Tumor by Co-Administration of Cancer Immunotherapeutic Agent aPD-1 and Akkermansia muciniphila EB-AMDK39 Live Cells in Syngeneic Melanoma Mouse Animal Model

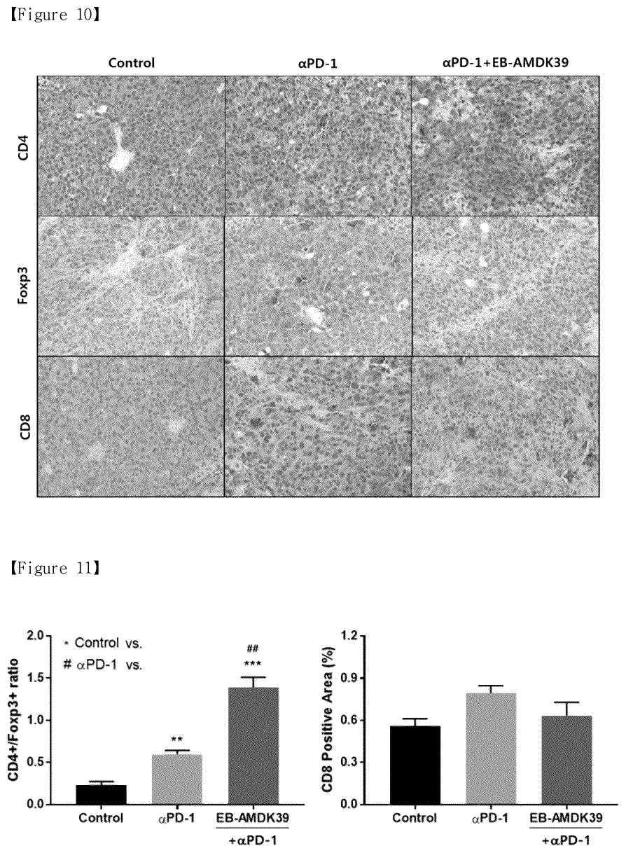

In order to examine the activity of immune cells in tumor tissue, CD4, Foxp3 and CD8 were analyzed by immunohistochemistry (IHC). Expression patterns were examined by microscopy, and analysis was performed using Image J image analysis software to measure the DAB-positive area appearing in yellowish brown to yellowish red. The positive areas (CD4, Foxp3, and CD8 positive cells) in each micrograph were measured and compared, and the results are shown graphically in FIGS. 10 and 11.

As shown in 10 and 11, as a result of examining the activity of immune cells in tumors by determining the ratio of CD4-positive T cells to Foxp3-positive Treg cells, it was confirmed that, in the aPD-1+EB-AMDK39-administered group, an increase in FoxP3-positive regulatory T cells was suppressed, and thus the CD4/Treg ratio was significantly higher than those in the other groups. This suggests that co-administration of aPD-1 and EB-AMDK39 lowers the Treg cell activity and increases the CD4 T cell activity. The above results show that, when the Akkermansia muciniphila EB-AMDK39 strain and the anti-PD-L1 antibody are co-administered, their effect on the activation of anti-tumor immune cells is further enhanced.

### Example 6: Anticancer Effect of Co-Administration of Cancer Immunotherapeutic Agent aPD-1 and Akkermansia muciniphila EB-AMDK39-Derived Extracellular Vesicles (EVs) in Syngeneic Melanoma Mouse Animal Model

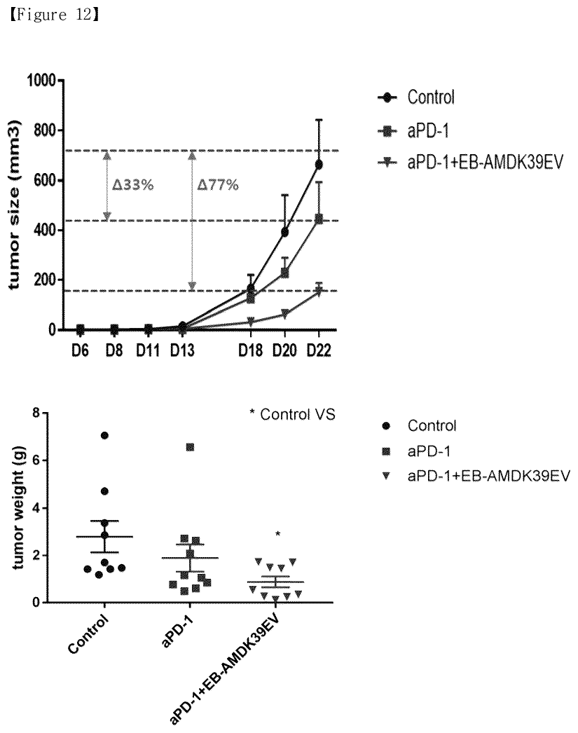

From day 6 when cancer cells began to appear, 250 μg of aPD-1 antibody and 100 μg of Akkermansla muciniphila EB-AMDK39 strain-derived extracellular vesicles (EVs) were intraperitoneally injected every 4 days. In this case, as the aPD-1 antibody, InVivoMab anti-mouse PD-1 (RMP1-14) (catalog #BE0146, BioX Cell) was used, which was diluted with InVivoPure pH 7.0 dilution buffer (catalog #IP0070) at a concentration of 250 μg/100 μl. The tumor size was monitored once every two days from the time the tumor started to appear, and the tumor size was calculated according to Equation 1 above, and the results are shown graphically in FIG. 12. The extracellular vesicles were tested for their efficacy in the mouse tumor model alone or in the presence or absence of anti-PD1.

Referring to FIGS. 12 and 13, it was confirmed that the tumor size became significantly smaller in the group to which the Akkermansla muciniphila EB-AMDK39 strain-derived extracellular vesicles (EVs) than in the control group into which the B16-F10 cells were syngeneically transplanted. Compared to the tumor size in the control group (664.4±178.8 mm³), the tumor size in the aPD-1-administered group was 445.3±148.1 mm³ (about 33% decrease), and the tumor size in the group to which PD-1 and the Akkermansia muciniphila EB-AMDK39 strain-derived extracellular vesicles (EVs) were co-administered was 150.7±37.52 mm³ (about 77% decrease), which significantly decreased (P<0.05).

As described above, the present inventors evaluated the cancer cell growth and metastasis inhibitory effects of the Akkermansia sp.-derived extracellular vesicles and the immune checkpoint inhibitor anti-PD1 in vivo, and as a result, found that, when the Akkermansia sp.-derived extracellular vesicles (EVs) and the immune checkpoint inhibitor anti-PD1 were injected into the syngeneic tumor mouse models, the tumor size and weight effectively decreased (see FIGS. 12 and 13).

Therefore, the pharmaceutical composition of the present invention has a significantly superior tumor growth inhibitory effect compared to anti-PD1 alone, and thus may be useful as a pharmaceutical composition for preventing or treating cancer.

### Example 7: Changes in Immune Cells in Tumors by Co-Administration of Cancer Immunotherapeutic Agent aPD-1 and Akkermansia muciniphila EB-AMDK39-Derived Extracellular Vesicles (EVs) in Syngeneic Melanoma Mouse Animal Model

To confirm the changes in immune cell activity in tumor tissue by co-administration of the cancer immunotherapeutic agent aPD-1 and the Akkermansia muciniphila EB-AMDK39-derived extracellular vesicles (EVs) in a syngeneic melanoma mouse animal model, immunohistochemical staining was performed.

In order to examine the activity of immune cells in tumor tissue, CD4, Foxp3 and CD8 were analyzed by immunohistochemistry (IHC).

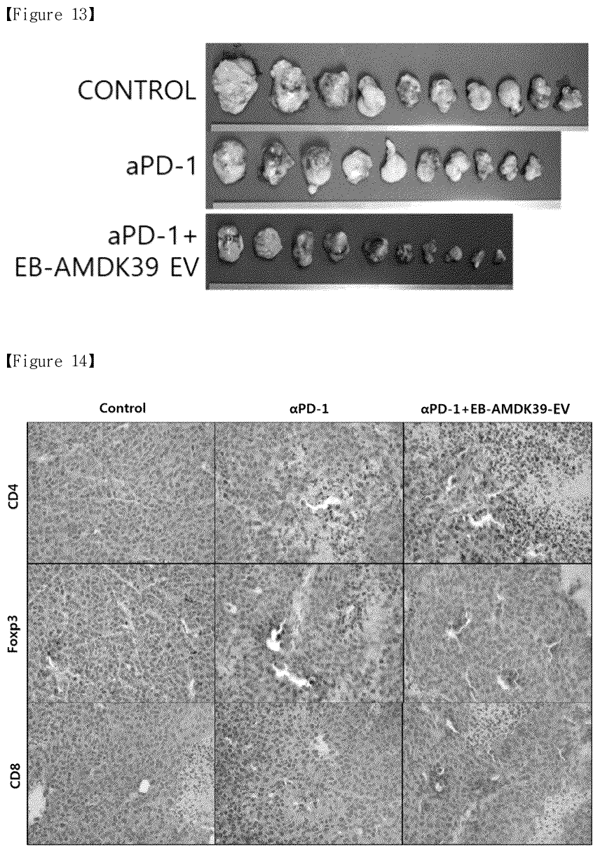

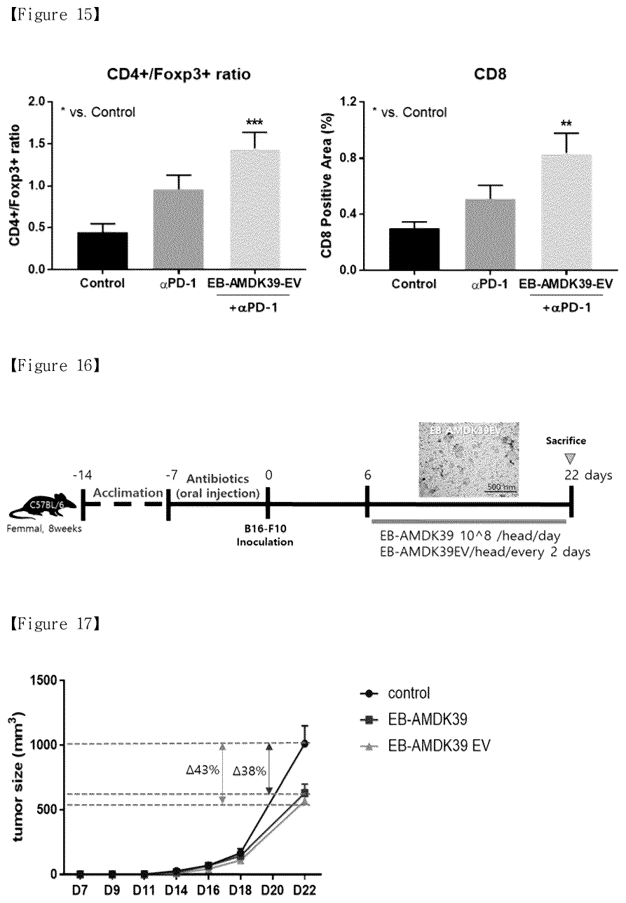

Immunohistochemical staining was performed on formalin-fixed/paraffin-embedded tissues using murine anti-CD4, anti-CD8 and anti-Foxp3 antibodies. Expression patterns were examined by microscopy, and analysis was performed using Image J image analysis software to measure the DAB-positive areas (CD4, Foxp3, and CD8 positive cells) appearing in reddish brown to reddish yellow. The positive areas in each micrograph were measured and compared, and the results are shown graphically in FIG. 14.

FIG. 14 shows the results of immunohistochemistry (IHC) of tumor cells after administration of the test substances. In FIG. 14, yellowish brown to yellowish red dots represent CD4, Foxp3 and CD8 positive cells.

As shown in 14 and 15, as a result of examining the activity of immune cells in tumors by determining the ratio of CD4-positive T cells to Foxp3-positive Treg cells, it was confirmed that, in the aPD-1+EB-AMDK39-administered group, an increase in FoxP3-positive regulatory T cells (Treg) was suppressed, and thus the CD4/Treg ratio was significantly higher than those in the other groups. In addition, the expression of cytotoxic T cells was analyzed by examining the CD8-positive area. It was confirmed that CD8 was also activated by CD4 T cells, and the CD8-positive area significantly increased in the group to which aPD-1 and EB-AMDK39EV were co-administered. Increased CD8 in tumor tissue means direct destruction of tumor cells.

### Example 8: Evaluation of Anticancer Effect of Akkermansia muciniphila EB-AMDK39 Live Cells or EVs

FIG. 16 schematically shows an in vivo experimental schedule for evaluating the anticancer effect of administration of Akkermansia muciniphila EB-AMDK39 live cells or EB-AMDK39 alone in a syngeneic melanoma mouse model.

After one week of acclimation, the antibiotics shown in Table 10 below were orally administered to C57BL/6 mice (female, 8 weeks old) for one week.

TABLE 10

| Antibiotic | Concentration |
|---|---|
| Ampicillin (Sigma-A0166) | 1 g/L |
| Vancomycin (Sigma SBR00001) | 0.5 g/L |
| Metronidazole (Sigma M1547) | 1 g/L |
| Neomycin (Sigma N6386) | 1 g/L |
| Amphotericin B (Sigma PHR1662) | 0.1 g/L |

2×10⁴ B16-F10 cells were subcutaneously (SC) injected into the thigh of each mouse together with 100 μl of Matrigel. From day 6 when cancer cells began to appear after B16F10 cell transplantation, EB-AMDK39 was orally administered daily in an amount of 10⁸ CFU/head/day or EB-AMDK39 EVs were intraperitoneally administered three times a week every other day in an amount of 100 μg/head. From the time the tumor appeared, the tumor size was measured using a caliper every two days, and the tumor size (volume) was calculated, and the results are shown graphically in FIG. 17.

As shown in FIG. 17, it was confirmed that the tumor size became significantly smaller in the group to which the Akkermansia muciniphila EB-AMDK39 was orally administered and the group to which the EB-AMDK39 EVs were intraperitoneally administered than in the control group into which B16-F10 cells were syngeneically transplanted. When comparing the tumor size on day 22, the tumor size in the Akkermansia muciniphila EB-AMDK39-administered group was 629±148.1 mm³, which decreased by about 30% compared to the tumor size in the control group (1,064±140.3 mm³) (P<0.05). Meanwhile, the tumor size in the Akkermansia muciniphila EB-AMDK39 EV-administered group was 568±77.71 mm³, which significantly decreased by about 43% (P<0.05).

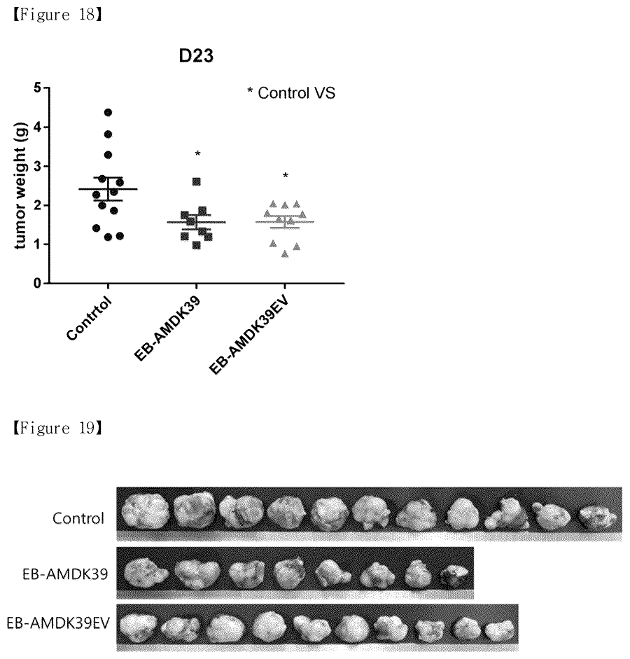

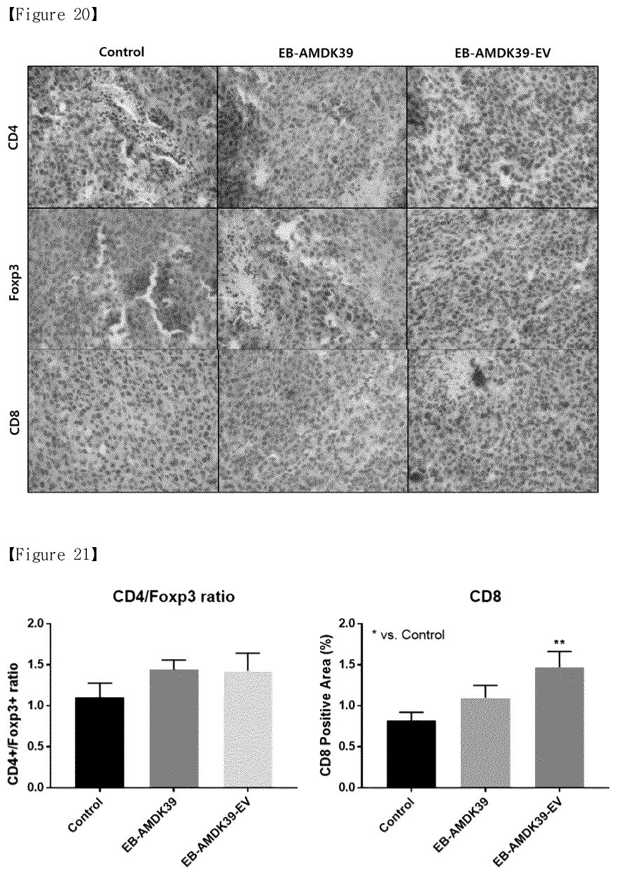

At the end of the animal model experiment, the mice were sacrificed on day 22, and the tumors were harvested and weighed, and the results are shown in FIG. 18. FIG. 19 shows images of tumor cells isolated from the mice. It was confirmed that the tumor weight in the control group was 2.423±0.2907 g, and the tumor weights in the Akkermansia muciniphila EB-AMDK39-administered group and the EB-AMDK39 EV-administered group were measured to be 1.564±0.1844 g and 1.573±0.1517 g, respectively, which significantly decreased (both P<0.05). As shown in FIG. 17, the tumor size significantly decreased when the Akkermansia muciniphila EB-AMDK39 strain or EB-AMDK39 EVs were administered alone.

### Example 9: Changes in Immune Cells in Tumor by Administration of Akkermansia muciniphila EB-AMDK39 Live Cells or EVs Alone

To confirm the changes in immune cell activity in tumor tissue by administration of the Akkermansia muciniphila EB-AMDK39 strain or the Akkermansia muciniphila EB-AMDK39 strain-derived extracellular vesicles (EVs) alone in a syngeneic melanoma mouse animal model, immunohistochemical staining was performed.

In order to examine the activity of immune cells in tumor tissue, CD4, Foxp3 and CD8 were analyzed by immunohistochemistry (IHC).

Immunohistochemical staining was performed on formalin-fixed/paraffin-embedded tissues using murine anti-CD4, anti-CD8 and anti-Foxp3 antibodies. Expression patterns were examined by microscopy, and analysis was performed using Image J image analysis software to measure the DAB-positive areas (CD4, Foxp3, and CD8 positive cells) appearing in reddish brown to reddish yellow. The positive areas in each micrograph were measured and compared, and the results are shown graphically in FIG. 21.

As shown in FIGS. 20 and 21, as a result of examining the activity of immune cells in tumors by determining the ratio of CD4-positive T cells to Foxp3-positive Treg cells, it was confirmed that the ratio of CD4 positive T cells to Foxp3 positive Treg cells in the group to which the Akkermansla muciniphila EB-AMDK39 strain or the EB-AMDK39 EVs were administered alone was slightly higher than that in the control group. As a result of analyzing cytotoxic T cells by examining CD8 positive T cells, it was confirmed that cytotoxic T cells were significantly increased by EB-AMDK39-EVs. These results suggest that the Akkermansia muciniphila EB-AMDK39 strain-derived extracellular vesicles (EVs) according to the present invention contribute to cancer treatment by activating anticancer immunity through increased intratumoral infiltration of T cells positive for CD8, which is a representative biomarker in anticancer mechanisms by cancer immunotherapeutic agents.

### Example 10: Confirmation of Reduced Metastasis Using Wound Healing Assay

HT29 human colorectal cancer cells were cultured in McCoy's medium containing 10% FBS and 1% gentamicin at 37° C. under 5% CO₂. HT29 colorectal cancer cells were seeded in a 6-well plate for cell culture and cultured confluently. Thereafter, the 6-well plate was uniformly scratched using a pipette tip. Next, the cells were treated with 1 or 10 μg/ml of EB-AMDK39 EVs for 24 hours and observed under a microscope. The cell area was calculated using the Image J program.

In order to evaluate the anticancer activity of EB-AMDK39 EVs, a wound healing activity test was performed. HT29 human colorectal cancer cells were wounded and then treated with 1 or 10 μg/ml of the Akkermansia sp.-derived extracellular vesicles (EVs) for 24 hours, and the extent of metastasis of the cancer cells was examined.

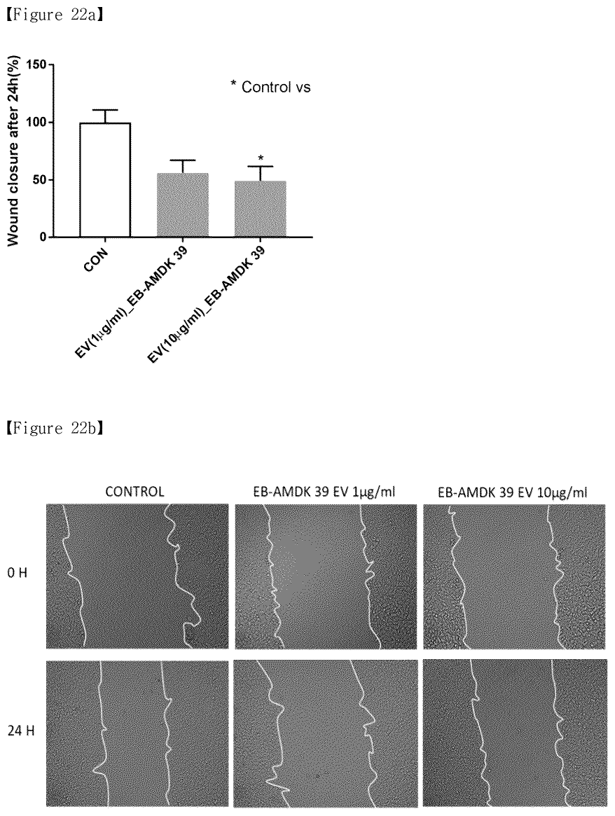

As shown in FIGS. 22a and 22b, as a result of the wound healing assay performed using HT29 cells, it was observed that the extents of the cancer cells in the groups to which 1 μg/ml and 10 μg/ml of the Akkermansia sp. strain-derived extracellular vesicles (EVs) were administered, respectively, decreased by 43.73% and 50.63%, respectively, compared to that in the control group. In particular, it was confirmed that the extent of metastasis significantly decreased in the group treated with 10 μg/ml of the extracellular vesicles (P<0.05).

### Example 11: Evaluation of Anticancer Efficacy in HT29 Cells

Since cytokines and other immunomodulators are involved in the regulation of inflammatory responses in inflammatory bowel disease, the present inventors examined whether the expression of these genes was affected by administration of the strain of the present invention. For an in vitro experiment for evaluation of anti-inflammatory efficacy, human colorectal epithelial HT-29 cells (ATCC HTB-38™, USA) were cultured. The cells were cultured using McCoy's 5A modified medium (Gibco, USA) supplemented with 10% FBS (fetal bovine serum, Hyclone, USA) and 10 μg/ml gentamicin as a basal medium in an incubator (NUAIRE, USA) at 37° C. under 5% C02. In order to examine whether the Akkermansia sp.-derived extracellular vesicles (EVs) inhibit the LPS-induced expression of IL-8 gene, a pro-inflammatory cytokine, in HT-29 cells, real-time PCR was performed using the primers in Table 11 below.

Total RNA was extracted using TRIZOL reagent (Sigma, USA), and 1 μg of RNA was synthesized into cDNA using the M-MLV cDNA synthesis kit (Enzynomics, Korea). Real-time PCR was performed using the Quant Studio 3 real-time PCR system (Applied Biosystems, USA).

Expression of the inflammatory cytokine gene was analyzed using the SYBR Green TOPreal™ qPCR 2× PreMIX (Enzynomics, Korea), and GAPDH was used as an internal standard. PCR was performed under the following conditions: pre-incubation (for UDG) at 50° C. for 4 min and 95° C. for 10 min, and 40 cycles, each consisting of 95° C. for 15 sec and 60° C. for 1 min. Data was analyzed by delta CT method using a program built in QuantStudio Design & Analysis Software v1.4.3.

TABLE 11

| Target | Direction | Primer sequence | SEQ ID NO. |
|---|---|---|---|
| GAPDH | F | 5′-GAC ATC AAG AAG GTG GTG AAG CAG-3′ | SEQ ID NO: 8 |
| | R | 5′-ATA CCA GGA AAT GAG CTT GAC AAA-3′ | SEQ ID NO: 9 |
| IL-8 | F | 5′-TTT TGC CAA GGA GTG CTA AAG A-3′ | SEQ ID NO: 10 |
| | R | 5′-AAC CCT CTG CAC CCA GTT TTC-3′ | SEQ ID NO: 11 |

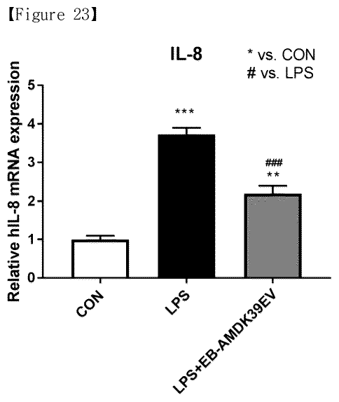

As shown in FIG. 23, it was confirmed that, when HT29 cells were treated alone with LPS (100 ng/ml) for 6 hours, the expression of the representative inflammatory cytokine IL-8 in the cells increased by about 3.7 times compared to that in the normal group (P<0.001). However, it was observed that the expression level of IL-8 in the group treated with LPS together with 1 μg/ml of the Akkermansia sp.-derived extracellular vesicles (EVs) decreased by 41% compared to that in the group treated with LPS alone (P<0.001).

The embodiments disclosed herein are only illustrative of preferred embodiments and are not intended to limit the scope of the present invention. It will be apparent to those skilled in the art that the present invention may be modified or altered in various forms without departing from the spirit and scope thereof. The scope of protection of the present invention should be defined by the appended claims, and the above modifications and variations are intended to fall within the scope of protection of the present invention.

### Microorganism Deposit Accession Number

Depository authority: Korean Collection for Type Cultures
Accession number: KCTC 13765BP
Deposit date: Dec. 5, 2018

1. A pharmaceutical composition for preventing or treating cancer or inflammatory disease containing extracellular vesicles (EVs) derived from an Akkermansia sp. and a pharmaceutically acceptable carrier, excipient, or stabilizer.

2. The pharmaceutical composition according to claim 1, wherein the Akkermansia sp. is an Akkermansia muciniphila.

3. The pharmaceutical composition according to claim 1, wherein the Akkermansia sp. is an Akkermansia muciniphila EB-AMDK39 strain (KCTC 13765BP).

4. The pharmaceutical composition according to claim 3, wherein the extracellular vesicles derived from the Akkermansia muciniphila EB-AMDK39 strain have an average diameter of 20 to 300 nm.

5. The pharmaceutical composition according to claim 1, wherein the cancer is any one selected from the group consisting of colorectal cancer, lung cancer, small cell lung cancer, gastric cancer, liver cancer, blood cancer, bone cancer, pancreatic cancer, skin cancer, head or neck cancer, skin or intraocular melanoma, uterine cancer, ovarian cancer, rectal cancer, perianal cancer, colon cancer, breast cancer, fallopian tube carcinoma, endometrial carcinoma, cervical cancer, vaginal cancer, vulvar carcinoma, Hodgkin's disease, esophageal cancer, small intestine cancer, endocrine adenocarcinoma, thyroid cancer, parathyroid cancer, adrenal cancer, soft tissue sarcoma, urethral cancer, penis cancer, prostate cancer, chronic or acute leukemia, lymphocytic lymphoma, bladder cancer, kidney cancer, ureteral cancer, renal cell carcinoma, renal pelvic carcinoma, CNS tumor, primary CNS lymphoma, spinal cord tumor, brainstem glioma, and pituitary adenoma.

6. The pharmaceutical composition according to claim 1, wherein the composition further contains a cancer treatment agent selected from a cancer chemotherapeutic agent and a cancer immunotherapeutic agent.

7. The pharmaceutical composition according to claim 6, wherein the cancer immunotherapeutic agent is at least one selected from the group consisting of anti-PD1, anti-PDL1, anti-CTLA, anti-Tim3, and anti-LAG3.

8. The pharmaceutical composition according to claim 1, wherein the composition further contains an Akkermansia sp..

9. The pharmaceutical composition according to claim 6, wherein the extracellular vesicles (EVs) derived from the Akkermansia sp. and the cancer chemotherapeutic agent or the cancer immunotherapeutic agent are administered simultaneously in a single dosage form, or administered simultaneously or sequentially in separate dosage forms.

10. A health functional food for preventing or ameliorating cancer containing extracellular vesicles (EVs) derived from an Akkermansia sp. and a nutraceutically or dietetically acceptable carrier or excipient.

11. A veterinary composition for preventing or treating cancer or inflammatory disease containing extracellular vesicles (EVs) derived from an Akkermansia sp. and a pharmaceutically acceptable carrier, excipient, or stabilizer.

12. An Akkermansia muciniphila EB-AMDK39 strain (KCTC 13765BP).


SEQUENCE LISTING (11 sequences)

SEQ ID NO: 1 - DNA, 1512 bp, unidentified
  misc_feature 1..1512: note = Akkermansia muciniphila
  source 1..1512: mol_type = genomic DNA
ttgatggagagtttgattctggctcagaacgaacgctggcggcgtggataagacatgcaagtcgaacgagagaattgctagcttgctaataattctctagtggcgcacgggtgagtaacacgtgagtaacctgcccccaagagtgggatagccccgggaaactgggattaataccgcataaaatcgcaagattaaagcagcaatgcgcttggggatgggctcgcgtcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtaaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctccttt

SEQ ID NO: 2 - DNA, 20 bp, synthetic construct
  misc_feature 1..20: note = AM1 primer (forward)
  source 1..20: mol_type = other DNA
cagcacgtgaaggtggggac

SEQ ID NO: 3 - DNA, 20 bp, synthetic construct
  misc_feature 1..20: note = AM2 primer (reverse)
  source 1..20: mol_type = other DNA
ccttgcggttggcttcagat

SEQ ID NO: 4 - DNA, 22 bp, synthetic construct
  misc_feature 1..22: note = ERIC-1 primer (forward)
  source 1..22: mol_type = other DNA
atgtaagctcctggggattcac

SEQ ID NO: 5 - DNA, 22 bp, synthetic construct
  misc_feature 1..22: note = ERIC-2 primer (reverse)
  source 1..22: mol_type = other DNA
aagtaagtgactggggtgagcg

SEQ ID NO: 6 - DNA, 20 bp, synthetic construct
  misc_feature 1..20: note = 27F primer (forward)
  source 1..20: mol_type = other DNA
agagtttgatcmtggctcag

SEQ ID NO: 7 - DNA, 20 bp, synthetic construct
  misc_feature 1..20: note = 1541 primer (reverse)
  source 1..20: mol_type = other DNA
aaggaggtgatccagccgca

SEQ ID NO: 8 - DNA, 24 bp, synthetic construct
  misc_feature 1..24: note = GAPDH primer (forward)
  source 1..24: mol_type = other DNA
gacatcaagaaggtggtgaagcag

SEQ ID NO: 9 - DNA, 24 bp, synthetic construct
  misc_feature 1..24: note = GAPDH primer (reverse)
  source 1..24: mol_type = other DNA
ataccaggaaatgagcttgacaaa

SEQ ID NO: 10 - DNA, 22 bp, synthetic construct
  misc_feature 1..22: note = IL-8 primer (forward)
  source 1..22: mol_type = other DNA
ttttgccaaggagtgctaaaga

SEQ ID NO: 11 - DNA, 21 bp, synthetic construct
  misc_feature 1..21: note = IL-8 primer (reverse)
  source 1..21: mol_type = other DNA
aaccctctgcacccagttttc